(19) United States
(12) Patent Application Publication
(10) Pub. No.: US 2008/0058958 A1
(43) Pub. Date: Mar. 6, 2008

(54) KNEE JOINT WITH RETENTION AND CUSHION STRUCTURES

(76) Inventor: Chia Pao CHENG, Shu Lin City (TW)

Correspondence Address: LEONG C LEI, PMB # 1008, 1867 YGNACIO VALLEY ROAD, WALNUT CREEK, CA, 94598, US

(21) Appl. No.: 11/749,163

(22) Filed: May 16, 2007

(30) Foreign Application Priority Data
Sep. 6, 2006 (TW) 095210040

(51) Int. Cl. A61F 2/62

(52) U.S. Cl. 623/39

(57) ABSTRACT

The present invention provides a knee joint including a joint top, a connection device, a cushion device, a resilient device, and a pneumatics joint body. The joint top and joint body are respectively joined to a thigh and a shank. With the cushion components including a connector and a connector, in the operation of kicking and swinging of leg in walking, standing, sitting, and kneeling, the angle between the connector and the connector support is changed to allow the knee joint to stay in a stable position and further preventing the shank from falling and/or kneeling during a walking operation and further effecting shock absorption by the cushion device at the time when the knee joint bends in walking along a slope, or setting the bending position of the knee joint with change of pressure applied to protect the user from the risk of abrupt kneeling.

### BACKGROUND OF THE INVENTION

(a) Technical Field of the Invention

The present invention relates to a retention and cushion structure of a knee joint, and in particular to knee joint shock absorption structure that may absorb impact forces by setting a connector and a connector support at a predetermined range of bending angle when the knee joint carries out actions.

(b) Description of the Prior Art

Medical technology and therapeutics are increasingly improved day by day and the disabled can be offered with increased moveability of walking through prosthetic devices, together with proper rehabilitation. The prosthetic device or artificial joint is often of a mechanical structure, which should be of a design that to the best extent mimics the action and configuration of human joints and should be set in the attempt to simulate the design of natural joint of human being in both shape and size to best satisfy the walking capability offered by the nature.

The convention artificial joint, as being pointed out in the disclosure of Taiwan Patent Publication No. 549074, is constructed in such a way that an upper end of the knee joint is formed to join to the tight of a disabled person and a lower end thereof connected to a shank and foot. Such a construction is generally formed of a compound linkage to provide the effect of simulating the natural knee joint and the lower link is provided with a cushion pad. When an upper acting link and a cushion link arranged at lower side are put in a line-like inward orientated direction, a drawback in operation of the known device is clearly observed, which will be explained as follows.

When the known knee joint is put into contact with ground in a straight stretching condition and thus receives an impact force, although the acting link may abut against the cushion pad to effect absorption of the impact force acting in the direction of the foot, yet the shock absorption function thereof can only be effected in the condition that the foot and the knee joint are substantially put in a line. For walking on a horizontal surface, the knee joint can be put onto contact with the ground in a substantially right-angle posture by properly lifting the leg to effect shock absorption. However, when walking in a downhill slope, to put the foot onto the ground in a natural way of walking, the knee joint has to be presented in small angle bending under which condition the known device apparently is not effective in fully exploiting the shock absorption capability thereof. Further, the links cannot be set and retained so that walking on slope may lead to the risks of abrupt bending or kneeling of the knee joint and inconvenience of walking in a slope for the disabled.

Thus, the present invention is aimed to improve the cushion structure of the conventional knee joint in order to present more natural way of walking through the knee joint cushion device of the present invention.

### SUMMARY OF THE INVENTION

In view of the above discussed drawbacks of the conventional prosthetic knee joints, the present inventor recognizes that with the poor effect of cushioning and shock absorption provided by the conventional knee joints, shock absorption effected by the cushion device of the knee joint cannot be fully exploited in walking in a slope, often leading to dangerous actions of abrupt downward bending or kneeling, and attempts to provide an improved kneel joint structure to effectively realize retention, cushioning, and shock absorption in walking and standing. It is also expected that the present invention may benefit the limb-disabled. Thus, the present inventor, based on the years' experience and devotion to the development of artificial limbs and prosthetic devices, creates the knee joint having retention and cushion structures that will be further discussed herein as the subject invention of the application.

An objective of the present invention is to provide a knee joint with retention and cushion structures, wherein when the knee joint bends rearward to carry out kicking and swinging of shank in walking or the knee joint is in standing, the angle of a connector is changed by load to abut against a connector support to set the knee joint at a stable position and protecting the shank from risks of falling or kneeling and, in addition, when walking in a slope, the angle of the knee joint after bending allows the cushion device to effect cushioning, and further, the knee joint can be changed by load to a stable position of angled bending to protect a user from the risk of kneeling down.

To achieve the above objective, the present invention provides a knee joint with bending cushioning structure, which comprises in construction, a joint top, a connection device (including two curved plates and a connector), a cushion device (including a connector support and a pneumatic cylinder), a resilient device (including spring pistons and springs), and a pneumatics joint body, which are assembled together with threaded connection. The joint top functions to join to a thigh located thereabove, and the pneumatics joint body that is located below is to connect to a shank and a foot therebelow. By means of the arrangement of cushion components on the connector support, the knee joint is provided with two fixed returning points, which allows the knee joint to effect bending, while in kicking and/or swinging of shank of walking and/or in standing, no excessive swinging is allowed between a connector and the connector support to exhibit a fixed stable condition, and which allows the installed knee joint to walk stably and allows the cushion device to effect cushioning and shock absorption by means of the rearward bending angle of the knee joint when walking in a slope to thereby make the disabled walking in a more comfortable and natural manner.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an exploded view of the knee joint constructed in accordance with the present invention;

FIG. 2 is a perspective view of the knee joint of the present invention in a partially assembled condition;

FIG. 3 is a cross-sectional view of the knee joint of the present invention illustrating the knee joint at a give position;

FIG. 4 is a side elevational view of the knee joint of the present invention at the give position;

FIG. 5 is a side elevational view of the knee joint of the present invention illustrating the knee joint is moved to another position;

FIG. 6 is a cross-sectional view of the knee joint of the present invention at said another position;

FIG. 7 is a side elevational view of the knee joint of the present invention illustrating the knee joint is moved to a further position;

FIG. 8 is a cross-sectional view of the knee joint of the present invention at said further position and demonstrating cushion effect of the knee joint;

FIG. 9 is a side elevational view of the knee joint of the present invention illustrating the knee joint is moved to yet a further position;

FIG. 10 is a cross-sectional view of the knee joint of the present invention at said yet a further position and demonstrating cushion effect of the knee joint;

FIG. 11 is a side elevational view of the knee joint of the present invention illustrating the knee joint is moved to yet another further position; and

FIG. 12 is a cross-sectional view of the knee joint of the present invention at said yet another further position and demonstrating cushion effect of the knee joint.

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

The present invention relates to a knee joint with retention and cushion structures, which comprises, in construction, a joint top 10, a connection device (including curved plates 30 and a connector 20), a cushion device (including a connector support 40 and a pneumatic cylinder 51), a resilient device (including spring pistons 52 and springs 53), and a pneumatics joint body 50, which are assembled by threading connection. The joint top functions to join to a thigh located thereabove, while the pneumatics joint body that is located below is to connect to a shank and a foot therebelow, wherein:

the joint top 10 has two projecting flange wings 11 extending from one side of a disk surface thereof and also forms an inwardly recessed opening that is between the two projecting flange wings and that is inwardly inclined and a semi-circular slot formed above the inwardly recessed opening to allow the inclined face to mate and join the connector 20 of the connection device, and the two sideway located projecting flange wings 11 of the knee top each form a through hole 12, so that with the connector forming, at a location corresponding to the inclined face of the inwardly recessed opening of the joint top, an inclined face for abutting purposes, and with the connector forming, at an upper portion thereof, an upper through hole 21 for joining, in an embedded manner, the joint top and received in the inwardly recessed opening of the joint top, the joint top and the connector each is provided with a resilient block 14 arranged on the inclined face thereof for abutting engagement and are jointed together as a unitary device by sideway fitting roller bearings 60 in the through holes of the projecting flange wings of the joint top and extending a shaft 13 through the upper through hole of the connector;

the block that constitutes the connector 20 forms, at a lower portion thereof, another, lower through hole 22 that is received between and connected with rounded dome projections 41 extending from opposite sides of an upper end of the connector support 40, and also forms, at a front side thereof, two lugs 23 defining at centers thereof connection through-holes 24 and receiving therebetween an inverted Y-shaped pneumatic cylinder driving rod 513 with a pin 25 extending therethrough to connect the pneumatic cylinder driving rod that contains therein a roller bearing 61 and the pneumatic cylinder driving rod is further coupled to a piston-rod joint 511 of a piston rod 512 of the pneumatic cylinder 51 that is concealed in the joint body 50, wherein the coupling between the connector and the connector support is provided by a pin 26 connecting through holes 42 defined in the sideways-located dome projections of the connector support and the lower through hole of the connector in which a needle bearing 60 is contained;

the connector support 40 is arranged in a cavity defined between extension wings 54 of the pneumatics joint body 50 and is provided, at a location below the dome projections thereof, with a through bore 43 that is in alignment with through holes 55 defined in the extension wings with a shaft 44 extending through the holes 55 that contain needle bearings 60 therein for engagement with the through bore so as to couple the connector support and the pneumatics joint body together, wherein the dome projections are put in abutting engagement with the spring pistons 52, so that the connector support can be subjected to constraints imposed thereon by the connector to have the dome projections abutting against the spring pistons by threadingly mounting an adjusting bolt 45 to a lower portion thereof and having the adjusting bolt 45 abutting against the joint body, causing compression of the springs 53 arranged below the spring pistons, which maintains the spring pistons in a resiliently biased condition to engage the connector support, whereby the connector support is resiliently supported and thus having cushion function; and

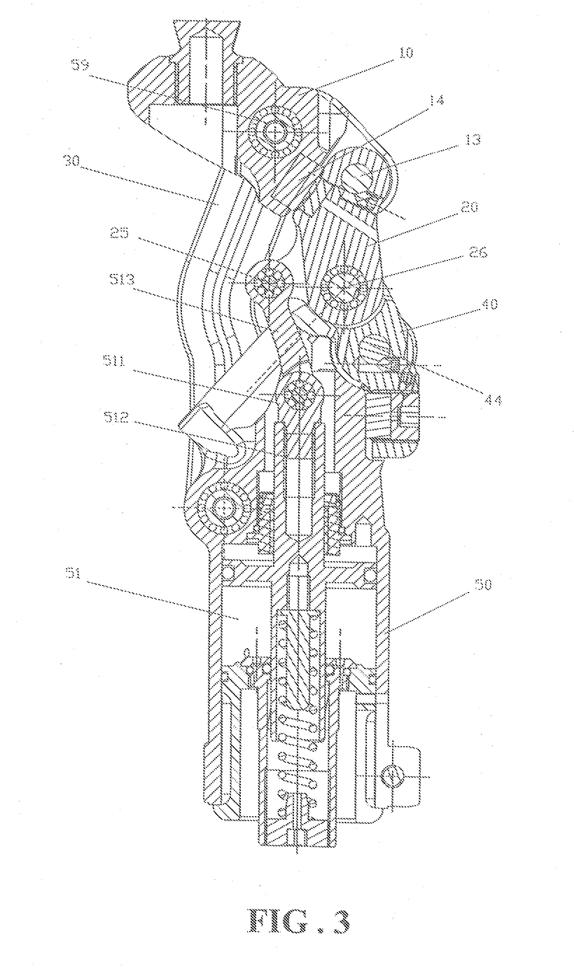

the pneumatics joint body 50 that is arranged at a lower side to connect to a shank and a foot therebelow is provided with the pneumatic cylinder 51 of the cushion device and joint-body tubular members 56 each having an open end closed and locked by a spring adjusting bolt 57 and an opposite open end into which the spring 53 and the spring piston 52 are fit so as to fix on the joint body, with the spring piston abutting against the connector support 40, and the joint body is provided in a symmetric manner at opposite sides thereof with through holes 58 in which needle bearings 60 are respectively received with an internally-threaded tube 59 extending through central bores of the needle bearings, and each curved plate 30 of the connection device is fixed, at one end thereof, to the internally-threaded tube by a hexagon socket bolt 62 and is also fixed, at an opposite end thereof, in the similar way to an internally-threaded tube that is fit into a central bore of a needle bearing mounted in a through hole 15 defined in one side face of the joint top 10 by a hexagon socket bolt, allowing the curved plate to rock along with the bending action of knee joint, whereby a knee joint cushion structure, which is particularly illustrated in the cross-sectional view of FIG. 3, is thus constructed by assembling the above-described components.

With reference now to FIG. 3, the assembling and operation of the components of the present invention will be described. By following the above described process, the connector 20 is matingly fit into the inwardly recessed opening of the joint top 10. The resilient block 14 is disposed on the inclined surface of the inwardly recessed opening. The piston-rod joint 511 is mounted to the piston rod 512 of the pneumatic cylinder 51 that is arranged inside the joint body and the inverted Y-shaped pneumatic cylinder driving rod 513 is fit so that the pneumatic cylinder driving rod is located between the lugs 23 of the connector and is secured by the roller bearing 61 and the pin 25 and the lower through hole of the connector is aligned with the through holes 42 of the dome projections 41 that are located on opposite sides of the connector support with the pin extending therethrough. The lower through hole 43 of the connector support cooperates with the shaft 44 to joint with the through holes 55 defined in the extension wings 54 of the pneumatics joint body. The through hole 15 that is defined in the joint top and the through hole 58 of the pneumatics joint body, after having been fit with the needle bearings and receiving the internally-threaded tubes therein, are jointed to each other by the curved plates 30 on the opposite sides and are secured by the flat-headed hexagon socket blots 62. The pneumatics joint body forms, at a lower edge thereof, an internally recessed bore for jointing the shank and the foot and the internally recessed bore is provided with threading for being jointed with a bolt for fixing purposes. The spring pistons 52 arranged on the joint body are put in abutting engagement with the dome projections 41 of the connector support and the spring forces of the resilient device are adjusted by means of the spring adjusting bolts 57 that threadingly engage the ends of the tubular members 56 and adjusting screws 46 of the connector support.

The connector support 40: in case that a user is standing, the connector support is subject to load and downwardly depresses the springs of the resilient device to timely effect cushioning so as to make the user of a prosthetic device feel more comfortable.

Valve adjusting screw 514: the adjusting screws function to change gas flowrate inside the pneumatic cylinder to thereby effect adjustment of the speed of bending and stretching of the joint.

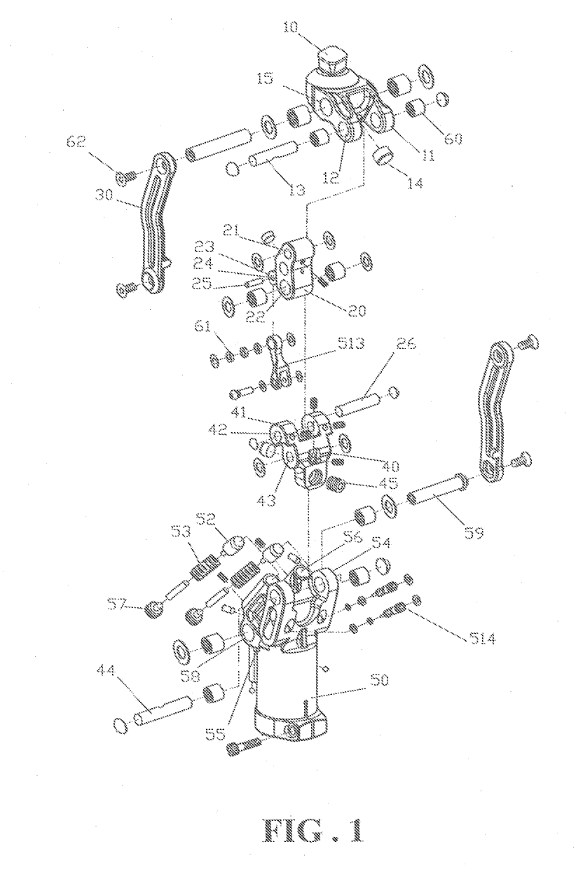

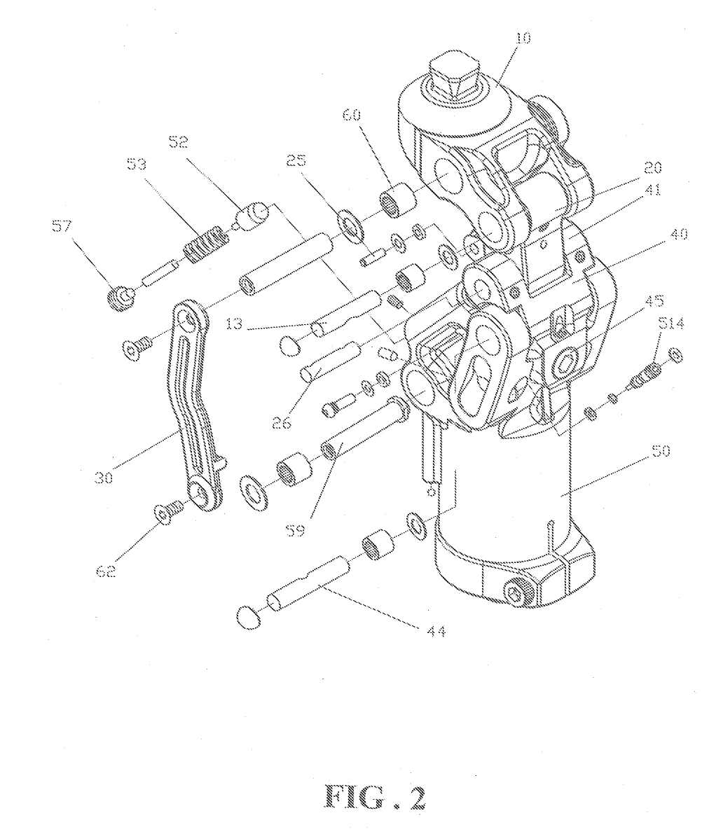

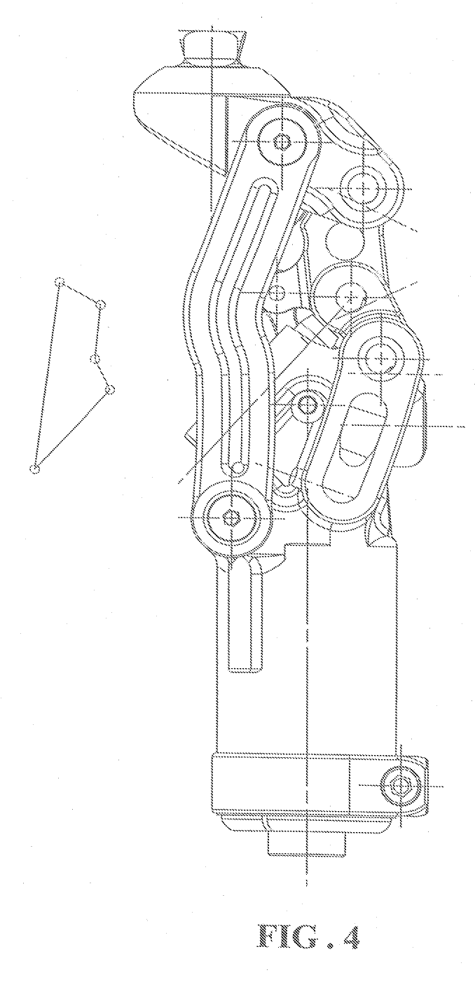

Referring to FIGS. 4-12, an operation of bending that is most preferably performed by the structure of the present invention will be described with reference to the drawings to explain the assembling of various components of the present invention with demonstration with simple steps, reference numerals being adopted the same as those shown in FIGS. 1-3.

As shown in FIGS. 4-12, the first step is a bending operation that the knee joint bends from a small angle to a large angle;

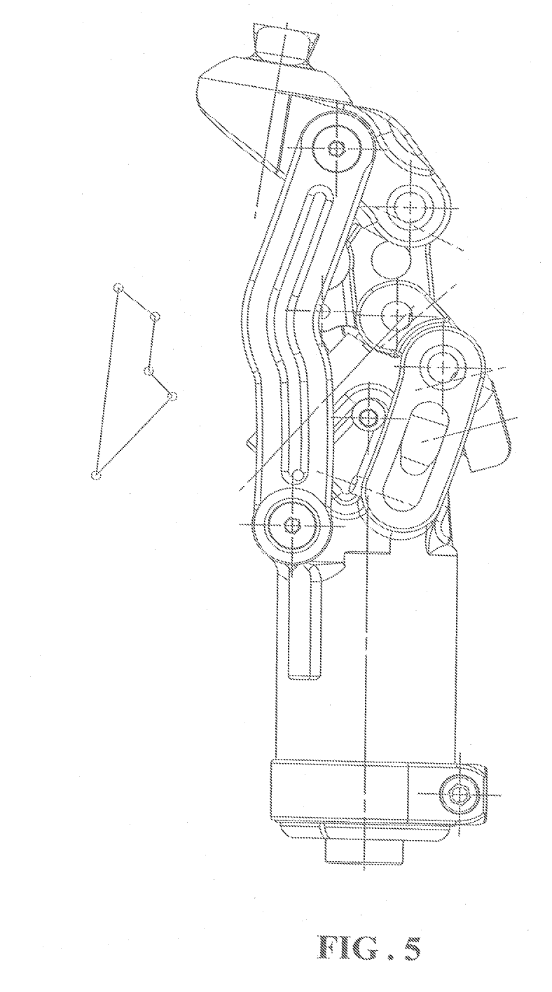

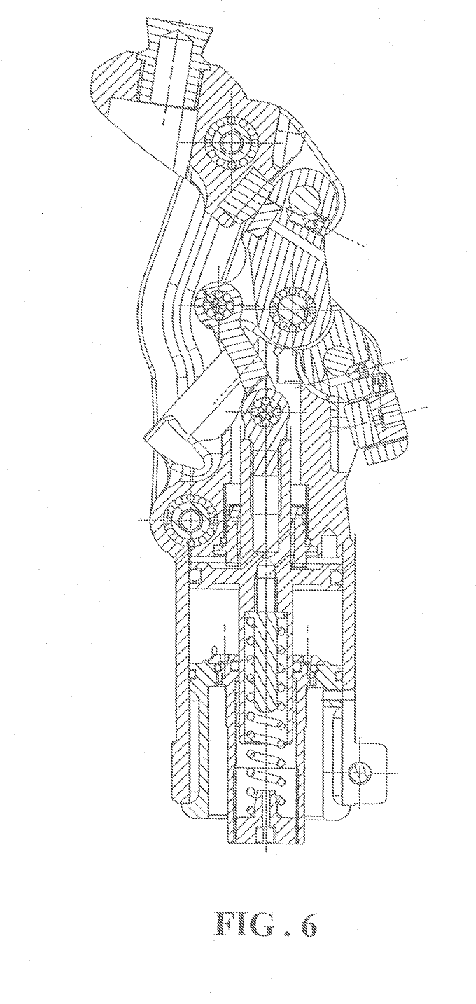

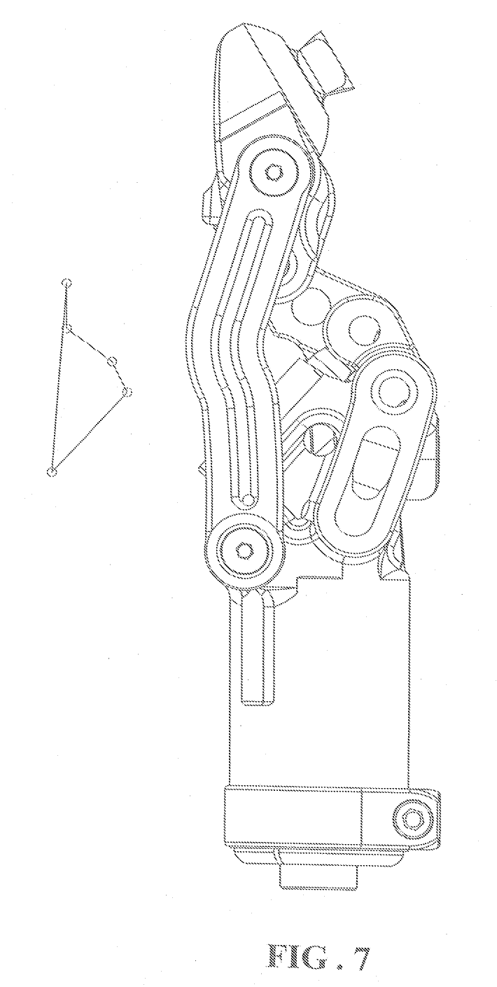

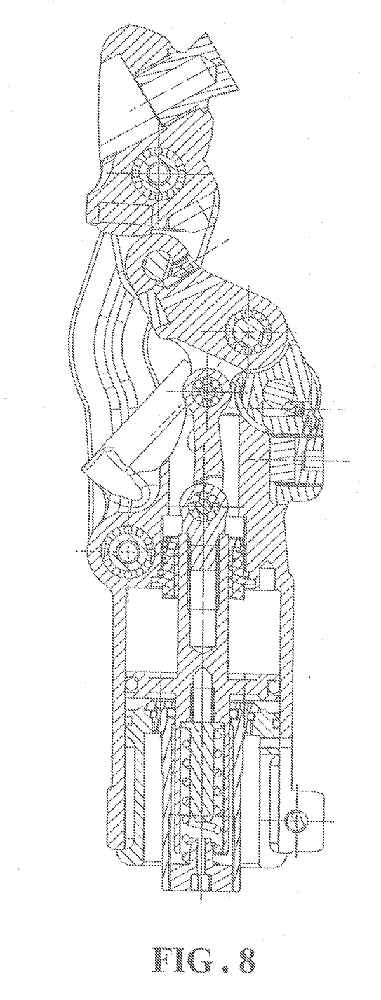

When the knee joint bends as the condition illustrated in FIGS. 5 and 6, where the joint top 10 is slightly bent to an angle of eight degrees, the connector 20 and the connector support 40 are subjected to a force of downward loading, causing a connection point between the connector and the joint top to displace downward and the connector support exhibiting a condition of downwardly depressing the tubular members 56 thereby effecting cushioning function; when the knee joint bends a greater angle, as illustrated in FIGS. 7 and 8, the joint top takes a condition of 62 degrees, where the curved plates 30 of the connection device are substantially parallel to the joint top, in which condition the pressures applied to the knee joint is the greatest and the connector 20 depresses the piston rod 512 to the lowest point. In a case that the joint top does not reach a 90-degree bent condition, the joint top under the above described bending situation, is subject to support by the pneumatic cylinder of the cushion device, as well as being abutted against and supported by the resilient device whereby the joint top of the knee joint can automatically return to a regular standing condition as illustrated in FIG. 4. Since the knee joint does not need to bend to a 90-degree condition in regular walking, the connector and the connector support, when performing kicking and swinging in doing a regular walking or standing, do not need to induce an excessive swinging therebetween and thereby exhibiting a fixed condition whereby risk of abrupt bending down or kneeling down occurring in the knee joint connector, when performing walking along a slope, can be eliminated. The knee joint can facilitates walking in a stable manner and can allow the cushion device to effect cushioning and shock absorption by means of the angle of bending of the knee joint in walking along a slope.

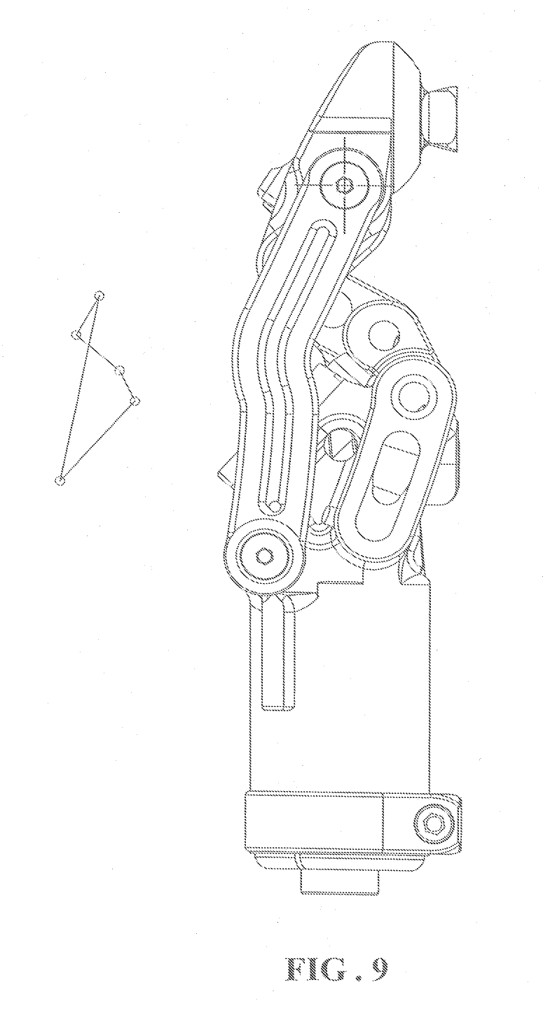

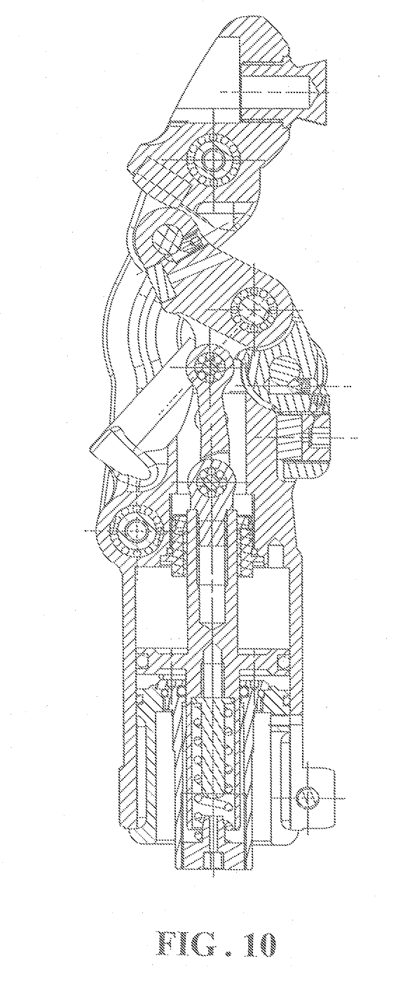

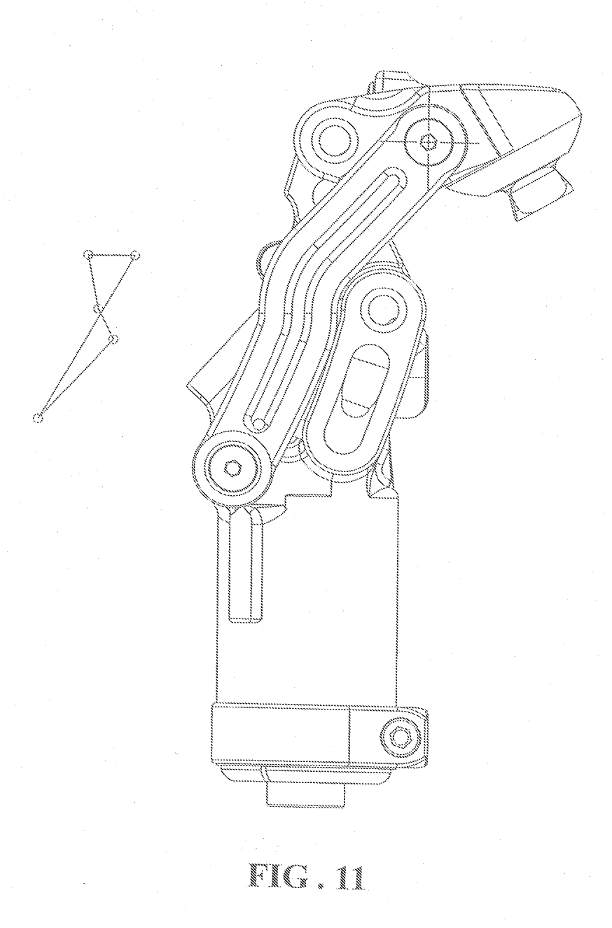

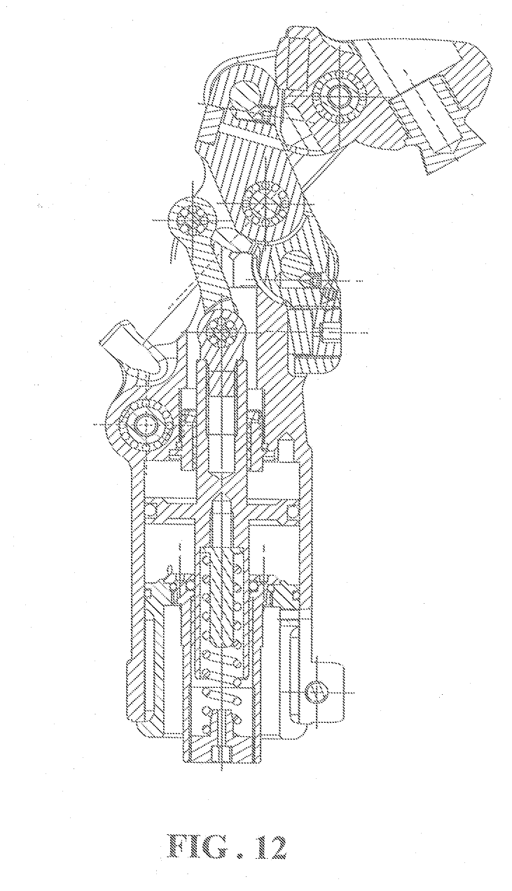

Also referring to the illustration of FIGS. 9 and 10, which demonstrate bending of the knee joint for further explanation of the present invention, as shown in these drawings, the knee top 10 is bent to 90 degrees, which allows a user to stably sit on for example a chair as an ordinary person, and the curved plates 30 are new located between the upper and lower through holes 21, 22 of the connector whereby when a force is applied to cause the bending of the joint top to become less than 90 degrees, the joint top of the knee joint, due to the support of the pneumatic cylinder of the cushion device and the abutting support by the resilient device, is automatically returned to the regular standing posture illustrated in FIG. 4 and when a force is applied to make the bending of the joint top exceeding 90 degrees, similarly due to the action of the cushion device and the resilient device, the inclined faces of the joint top and the connector, which are originally abut against each other, are changed by the rotation angle of the connector to such a condition that the inclined face is now in abutting engagement with a rounded top face of the connector so as to allow the joint top to bend toward another fixing point and the curved plates of the connection device is now moved to a location corresponding to the connector support, as illustrated in FIGS. 11 and 12, whereby the joint top is allowed to bend an angle of 150 degrees and the user is thus allowed to kneel down for carrying out on-ground operations by means of the bending of the knee joint. It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A knee joint with retention and cushion structures, comprising: a joint top, a connection device, a cushion device, a resilient device, and a pneumatics joint body, which are assembled by threading connection, the joint top being adapted to join to a thigh located thereabove, the pneumatics joint body that is located below being adapted to connect to a shank and a foot therebelow, wherein:
the connector is fit in an inwardly recessed opening of the joint top and a resilient block is positioned on an inclined abutting face formed in the recessed opening, a pneumatic cylinder being arranged inside the joint body and having a piston rod having a front end coupled to a pneumatic cylinder driving rod, the pneumatic cylinder driving rod being arranged between lugs formed on the connector and connected thereto in a pin-extending-through-bearing manner, the connector forming a lower opening that is aligned with through holes defined in rounded dome projections formed on opposite sides of the connector support and is fixed by a retention pin extending therethrough, the connector body forming at a lower portion thereof a through bore that is connected to through holes defined in extension wings of the pneumatics joint body by a shaft, additional through holes being defined in both the joint top and the pneumatics joint body and receiving therein needle bearings through which internally-threaded tubes extend to opposite sides of which curved plates are respectively mounted by hexagon socket bolts, springs and spring pistons being arranged on the pneumatics joint body with the spring pistons abutting against the dome projections of the connector support, the pneumatics joint body forming, at a lower edge thereof, an internally recessed bore for jointing the shank and the foot, the internally recessed bore being formed with the threading for facilitating jointing with a bolt.
